(19) United States
(12) Patent Application Publication
(10) Pub. No.: US 2004/0138103 A1
(43) Pub. Date: Jul. 15, 2004

(54) Compositions containing peptide copper complexes and metalloproteinase inhibitors and methods related thereto

(75) Inventor: Leonard M. Patt, Seattle, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: ProCyte Corporation, Redmond, WA

(21) Appl. No.: 10/696,536

(22) Filed: Oct. 29, 2003

Related U.S. Application Data
(60) Provisional application No. 60/425,203, filed on Nov. 7, 2002.

(51) Int. Cl. A61K 38/16

(52) U.S. Cl. 514/6

(57) ABSTRACT

Novel compositions capable of inhibiting the degradation of extracellular matrices of warm-blooded animals, including humans, and promoting the production of proteins thereof, combine at least one metalloproteinase inhibitor, which may be a matrix metalloproteinase inhibitor, and at least one peptide copper complex. Also disclosed are methods that utilize the disclosed compositions, by administering to warm-blooded animals effective amounts thereof orally, parenterally, or topically, for treating arthritis and other inflammatory conditions, enhancing wound and bone healing, treating skin diseases, treating cosmetic defects of the skin, or stimulating hair growth.

### CROSS-REFERENCE TO RELATED APPLICATION

This application claims the benefit of U.S. Provisional Patent Application No. 60/425,203 filed Nov. 7, 2002, which provisional application is incorporated herein by reference in its entirety.

### BACKGROUND OF THE INVENTION

1. Field of the Invention

The present invention generally relates to compositions used for treating pathological conditions associated with abnormal activity of metalloproteinases and matrix metalloproteinases, and for reversing tissue damage associated therewith.

2. Description of the Related Art

There are a number of cosmetic defects, diseases, and other pathological conditions associated with the degradation of an extracellular matrix (hereinafter, “ECM”). An ECM, also referred to as connective tissue, is a complex structure surrounding and supporting tissue cells of warm-blooded animals. More specifically, an ECM is an aggregate of connective tissue proteins that interact to form a highly insoluble material. In this way, an ECM functions as the glue that holds cells together in tissues. ECMs play an important role in regulating cellular functions during both normal and pathological remodeling processes, such as embryonic development, tissue repair, aging, inflammation, tumor invasion, and metastasis. Typically, an ECM is composed of three major classes of biomolecules: 1) structural proteins, including collagen and elastin; 2) specialized proteins, such as fibrillin, fibronectin, and laminin; and 3) proteoglycans.

Collagens are the major protein component of an ECM and the major structural protein of the skin, accounting for more than 70% of the dry weight of skin. There are at least 12 types of collagen. Types I, II, and III are the most abundant and form fibrils of similar structure. Type IV collagen is the principal collagen constituent of most basal laminae (also called “basement layers”). A basal lamina is a layer that separates epithelia from the tissue beneath.

Elastin is the major protein component of the elastic fibers that impart an extensible and resilient character to tissues such as the dermis. Elastin is secreted from cells in the form of soluble tropoelastin monomers, and assembles with other microfibrillar components to form the elastic fiber.

Fibrillin actually occurs in two forms, fibrillin-1 and fibrillin-2. These are extracellular acidic proteins that have an extended, thread-like shape and are found throughout connective tissue as integral components of the non-collagenous, extended fibrils (also referred to as “microfibrils”) of ECMs. The latter occur both isolated and in conjunction with elastin and are abundant in skin, blood vessels, and tendons.

Fibronectin is a glycoprotein found in most ECMs as aggregates or fibrils. Its functions include cell adhesion, migration, and invasion. Laminin is a structural adhesion glycoprotein, found in all basal laminae, that anchors cell surfaces to the basal lamina.

Proteoglycans are non-collagens and are composed of a protein core having, covalently bonded thereto, one or more glycosaminoglycan (hereinafter, “GAG”) side-chains. Examples include chondroitin sulfate and heparan sulfate.

Key factors in the degradation of an ECM are metalloproteinases (hereinafter, “MPs”) particularly, those MPs referred to as matrix metalloproteinases (hereinafter, “MMPs”). MMPs include membrane-type MMPs (hereinafter, “MT-MMPs”). MPs belong to a superfamily of zinc-dependent proteases (proteolytic enzymes) known as metzincins. MMPs are a group of proteases that are zinc-binding endopeptidases that are involved in connective tissue matrix remodeling and degradation of ECMs, both as part of normal physiological processes and in pathological conditions. MMPs are capable of degrading a variety of ECM protein components, including: collagen, proteoglycans, fibronectin, and laminin.

There are five major groups of MMPs: 1) collagenases (MMP-1, MMP-8, and MMP-13); 2) gelatinases (MMP-2 and MMP-9); 3) stromelysins (MMP-3, MMP-10, and MMP-11); a heterogeneous subgroup that includes: matrilysin (MMP-7), enamelysin (MMP-20); macrophage metalloelastase (MMP-12), and MMP-19; and 5) MT-MMPs. MT-MMPs activate progelatinase A and a cascade of matrix proteases that, in turn, degrade the ECM. A comprehensive list of known MMPs, including their designation and corresponding names, as well as the substrates that they act upon, is given in the table below.

| Enzyme | Other names | Preferred substrates |
|---|---|---|
| MMP-1 | Collagenase-1, | Collagens I, II, III, VII, X, gelatins |
| | interstitial collagenase | |
| MMP-2 | Gelatinase A, 72 kDa | Gelatins, collagens IV, V, VII, X, |
| | gelatinase | elastin, fibronectin; activates pro- |
| | | MMP-13 |
| MMP-3 | Stromelysin-1 | Proteoglycans, laminin, fibronectin, |
| | | gelatins |
| MMP-7 | Matrilysin | Proteoglycans, laminin, fibronectin, |
| | | gelatins, collagen IV, elastin, |
| | | activates pro-MMP-1 and -2 |
| MMP-8 | Collagenase-2, | Collagens I, II, III |
| | neutrophil collagenase | |
| MMP-9 | Gelatinase B, 92 kDA | Gelatins, collagens IV, V, elastin |
| | gelatinase | |
| MMP-12 | Macrophage | Elastin, collagen IV, fibronectin, |
| | metalloelastase | activates pro-MMP-2 & 3 |
| MMP-13 | Collagenase-3 | Collagens I, II, III, gelatins |
| MMP-14 | MT-MMP-1 | Activates pro-MMP-2 & 13, |
| | | gelatins |
| MMP-15 | MT-MMP-2 | unknown |
| MMP-16 | MT-MMP-3 | Activates pro-MMP-2 |
| MMP-17 | MT-MMP-4 | unknown |

Ideally, the synthesis, activation, and inhibition of MMPs is tightly regulated at several levels to maintain a proper balance between the synthesis and breakdown of tissue. However, departure from this ideal often occurs and results in various manifestations of aging and cosmetic defects, disease states, and other pathological conditions.

Normal aging, stress, and environmental exposure, for example, can increase the activity of MMPs so as to accelerate the degradation of ECMs, the latter playing an important role in the aging process. Aging is accompanied by a gradual decrease in dermal thickness, the amount of collagen, and the degree of protein organization—all of which are essential for youthful-looking skin. The epidermis (outer skin layer) changes subtly with age, while the dermis (inner skin layer) shows more profound changes. Collagen becomes disorganized with broken fibers, and the ECM shows widespread destruction. Also, the collagen tends to cross-link with age, diminishing the elasticity and youthful tone of the skin. The population of fibroblasts is reduced by about 50% by the age of 80.

Fibroblasts play a leading role in the ongoing regeneration of the dermis. Their proper functioning depends upon a proper balance being struck between the destruction of extracellular proteins and the synthesis of new protein. In turn, this balance depends upon a proper balance between MMPs and their inhibitors. As a result, for example, of aging, wounding, inflammation, and environmental exposure, excessive amounts of MMPs may be excreted and destroy ECMs by breaking down collagen and other ECM components.

As one example, exposure to even small amounts of UV radiation can damage collagen fibers, cause an accumulation of abnormal elastin, and result in the production of abnormally large amounts of MMPs relative to their respective inhibitors that keep them in check. For example, when lightly to moderately pigmented skin is exposed to sunlight for 5 to 15 minutes, levels of MMPs remain elevated for about one week. The MMPs degrade collagen and yield an uneven ECM of disorganized collagen fibers. When this process is repeated, wrinkles form. Wrinkles caused by smoking have also been attributed to an imbalance of ECM-degrading MMPs and their respective inhibiting factors. Again, this imbalance results in degradation of the ECM, particularly of collagen, which in turn results in a loss of skin tone and, eventually, wrinkles.

Degradation of an ECM, largely by abnormally elevated activity of MPs and MMPs, is also involved in certain arthritic conditions. For example, osteoarthritis (hereinafter, “OA”) and rheumatoid arthritis (hereinafter, “RA”) are characterized by the destruction of the articular cartilage ECM (hereinafter, “the AC-ECM”), which is an ECM made up of fibril-forming collagens (mostly Type II), aggrecan, and many other important molecules.

In patients with OA and RA, elevated levels of MMPs (especially MMP-1, MMP-3 and MMP-13) have been found in the synovium (joint lining) and cartilage, suggesting their role in these diseases. Also, the regulation of MMPs has been found to be aberrant with these conditions. Based on such evidence, MPs and MMPs are regarded as playing a key role in joint articular tissue degeneration. More specifically, the progressive cartilage and bone destruction, characteristic of OA and RA, is considered to be driven by an excess of MP and MMP enzymes. In fact, MMPs are able to cleave all compounds that make up the cartilage matrix. Thus, excessive activity results in a breakdown of collagen to the point of causing joint damage.

ECM degradation is also involved in both normal and abnormal wound healing. Wound healing has three phases: 1) inflammation, 2) tissue formation, and 3) tissue remodeling. A necessary step in wound healing is degradation of the ECM. Cell movement into the affected ECM for healing may require an active proteolytic system for clearing a path. Such a system uses various fibroblast-derived enzymes, such as MMP-1 (collagenase), and serum-derived plasmin. The activation of collagenase, for example, leads to degradation of collagen and other ECM proteins. The degradation of collagen in a wound is controlled by several MMPs, which are secreted by macrophages, epidermal cells, endothelial cells, and fibroblasts.

Various phases of wound repair rely on distinct balances of MMPs and their respective inhibitors. When such balances are tipped in favor of the MMPs, abnormal wound healing may result. For example, overexpression and the associated activation of MMP-8 may be associated with the pathogenesis of non-healing, chronic leg ulcers. Also, diabetic ulcers—an example of abnormal wound healing—are characterized by prolonged inflammation, decreased synthesis of collagen, and increased levels of proteinases.

Overexpression and the associated activity of MPs and MMPs are also involved in inflammatory conditions and various skin diseases, such as psoriasis, eczema, and acne rosacea. Inflammatory disease is associated with the abnormal release, the latter being caused by MMPs, of pro-inflammatory cytokines and similar proteins. Also, an imbalance involving the release by MPs and MMPs of certain cytokines, certain growth factors, and matrix proteins, can result in the above-mentioned skin diseases, among others.

As the prior art is lacking in respect thereof, there is a need in the art for compositions, and methods related thereto, that are effective in both: 1) halting pathological ECM degradation and stemming the course of certain diseases that, along with such degradation, result from excessive activity of MPs and MMPs; and 2) reversing the effects of the excessive activity of MPs and MMPs by stimulating the production of ECM proteins, such as collagen, elastin and proteoglycan. The present invention fulfills these needs and provides further related advantages.

### BRIEF SUMMARY OF THE INVENTION

In brief, the present invention is directed to compositions having utility for inhibiting the degradation and promoting the production of ECM proteins; as well as to methods that use such compositions for cosmetically treating the skin of a warm-blooded animal to reduce the signs of aging and environmental exposure, and for treating, as well as repairing the damage caused by, various diseases and pathological conditions such as abnormal wound and bone healing, inflammation, arthritis, and certain skin diseases. A “warm-blooded animal,” as the expression is used herein, includes a human, and is hereinafter referred to as a “patient.”

In one representative embodiment, the present invention is directed to compositions that combine at least one peptide copper complex and at least one MP inhibitor. In another representative embodiment, the MP inhibitor is a MMP inhibitor. The MP and MMP inhibitors may be derived from natural sources or synthesized. As the compositions may be administered orally, parenterally, or topically, in additional representative embodiments, the composition of the present invention further includes an inert and physiologically acceptable carrier or diluent, a sunscreen agent, a skin conditioning agent, a skin protectant, an emollient, a humectant, an excipient, a thickening agent (textural modifier), an emulsifying agent, a preserving agent, or a mixture thereof. Also, the composition may be in the form of a liquid, a cream, a suspension, a gel, an emulsion, a lotion, or an oil.

In another representative embodiment of the disclosed composition, the at least one peptide copper complex and/or MP inhibitor comprised therein are encapsulated in a liposome or microsponge adapted to aid in the delivery of the peptide copper complex and/or MP, or to enhance the stability of the composition. The disclosed composition, in yet another embodiment, comprises at least one MP inhibitor and at least one peptide copper complex that are formulated in an instrument adapted to deliver the compounds via iontophoresis. In a related, particular embodiment, the at least one peptide copper complex and at least one MP inhibitor are formulated for delivery via ultrasound.

In another embodiment, the composition comprises at least one MP inhibitor and at least one peptide copper complex that are formulated for topical application after a treatment, such as a laser treatment, to remove or partially remove the stratum corneum to improve the transport and delivery of the active compounds to the skin.

The present invention is also directed, in further representative embodiments, to methods for treating arthritis and other inflammatory conditions, enhancing wound and bone healing, treating skin diseases, or treating cosmetic defects of the skin, by administering an effective amount of a composition of the present invention orally, parenterally, or topically.

These and other aspects of this invention will be evident upon reference to the following detailed description of the invention.

### DETAILED DESCRIPTION OF THE INVENTION

As noted above, in one representative embodiment, there is disclosed a composition that combines at least one peptide copper complex and at least one MP inhibitor. In another representative embodiment, there is disclosed a composition that combines at least one peptide copper complex and at least one MMP inhibitor. The compositions are in a form suitable for oral, parenteral, or topical administration to a patient, and are effective in treating diseases and other pathological conditions, as well as cosmetic defects, by both normalizing excessive proteolytic activity of MPs and MMPs, respectively, and repairing the damage caused thereby. Methods for so treating such diseases, conditions, and defects by administering the compositions to an affected patient, are also disclosed.

As used herein, the expression “abnormal activity of MPs” refers generally to degradation of an ECM via proteolytic cleavage by MPs of ECM proteins to an extent beyond what may be necessary as a step in a normal physiological process. An example of the latter is the normal process associated with wound healing. Generally, this abnormal activity results from an imbalance of the MP or MPs, involved in the ECM degradation, and their associated inhibitors. The expression “abnormal activity of MMPs,” (as noted, MMPs are an example of MPs) has a similar meaning.

Also, as used herein, the expressions “MP inhibitor” and “MMP inhibitor” refer to any agent that inhibits the activity and/or production of MPs and MMPs, respectively, so as to inhibit their ability to degrade ECMs or otherwise destroy proteins through proteolytic cleavage. Such inhibiting agents include those agents that are naturally produced by the body for the purpose of inhibiting MPs and MMPs, or naturally occurring in general, or synthetic.

In a particular embodiment of the composition disclosed herein, the MMP inhibitor comprised therein is a tissue inhibitor of MP (hereinafter, “TIMP”), recombinant TIMP, or a derivative thereof. In related, more specific embodiments, the TIMP is TIMP-1, TIMP-2, TIMP-3 and TIMP-4, respectively. TIMPs are a family of proteins that are natural, specific physiological inhibitors of MMPs and are synthesized by the same cells that produce MMPs. TIMP-1 to TIMP-4 are the four TIMP molecules that have been identified to date. All active MMPs are inhibited by TIMPs with a stoichiometric ratio of 1:1. Inhibition of the MMPs results from non-covalent bonding of the TIMP to the active site of the MMP and to the proforms of the gelatinases (MMP-2 and MMP-9).

The composition of the present invention, in another particular embodiment, comprises a MMP inhibitor that is α₂-macroglobulin. The latter is a large, naturally occurring protein (750 kDa) produced by the liver and found in the serum and synovial fluids of normal and OA patients. It is a non-specific inhibitor of MMPs that acts as such by trapping the proteases and blocking their access to the substrates that they are otherwise able to degrade through proteolytic cleavage.

The MMP inhibitor, in yet other embodiments is a macrocyclic lactone, a bisphosphonate, or certain inhibitors derived from hydroxamic acid. Macrocyclic lactones are naturally occurring and include Bryostatins. The latter have been shown to have both in vitro and in vivo activity against MMPs. They do not directly affect the activity of MMPs but, rather, inhibit their production. Bisphosphonates are a class of drugs currently in use for diseases of bone-resorption. These compounds have been shown to have MMP inhibitory activity in vitro, due possibly to their cation-chelating ability. They have also been shown to inhibit secretion of MMP-2.

The inhibitors derived from hydroxamic acid are generally a class of broad spectrum, small molecule synthetic inhibitors of MMPs that act as such by non-covalently chelating, so as to bind, the catalytic zinc atom found in the active site of the MMPs. One related embodiment is directed to a non-peptidic hydroxamte inhibitor that, in a more specific embodiment, is marimastat, a MMP inhibitor drug manufactured by British Biotech PLC, Oxford, England. Marimastat acts against MMP-1, MMP-2, MMP-3, MMP-7 and MMP-9. Another related embodiment is directed to a pipcolinic hydroxamic derivative, that, in a more specific embodiment, is pipcolinic sulfamide. These latter MMP inhibitors are manufactured by Agouron Pharmaceuticals, Inc., La Jolla, Calif.

In another embodiment of the present invention, the MMP inhibitor disclosed therein is a malony-α-mercaptoalcohol, a succinyl-α-mercaptoalcohol, a malony-α-mercaptoketone, or a succinyl-α-mercaptoketone. In yet another embodiment, the MMP inhibitor is an antibiotic that, in a more specific embodiment, is anthracycline, tetracycline, doxycycline, minocycline, or a derivative thereof. Anthracycline has been recognized for its ability to inhibit MMP activity. Tetracycline and its semisynthetic forms, doxycycline and minocycline, inhibit MMPs both in vitro and in vivo, and appear to be active against collagenases and gelatinases.

A composition of the present invention, in another specific embodiment comprises a MMP inhibitor that is a retinoid, a thyroid hormone, a glucocorticoid, progesterone, or an androgen. These compounds have been found to inhibit the synthesis of MMPs in many types of cells. As an example, the retinoid, N4-OH phynylretinamide, inhibits MMP-1 synthesis in synovial fibroblasts. The MMP, in a related embodiment, is the peroxisome proliferator-activated receptor gamma (PPARγ). Studies have suggested that PPARγ ligands participate in the control of inflammation by suppressing the production of pro-inflammatory cytokines, and have also shown that PPARγ inhibits the production of MMP-9 in macrophages, and play a role in the suppression of MMP-1 and MMP-13 (see, e.g., Fahmi, H. et al., “Peroxisome proliferator-activated receptor gamma activators inhibit interleukin-l-beta-induced nitric oxide and matrix metalloproteinase 13 production in human chondrocytes,” Arthritis & Rheumatism, 44: 595-607, 2001).

The MMP inhibitor, comprised in the composition disclosed herein, in another embodiment, is an antisense RNA or ribozyme. Such compounds can specifically affect the mRNA of a single MMP. For example, it has been shown that delivery of a stromelysin-specific ribozyme into the knee joints of rabbits reduced the MMP-3 expression in the synovium. As another example, an anti-MMP-2 ribozyme, introduced into glomerular mesangial cells, caused loss of the inflammatory phenotype. Finally, a MMP-9 antisense-ribozyme expression construct was shown to inhibit expression of MMP-9.

In yet another embodiment directed to compositions, the MMP inhibitor combined with the peptide copper complex comprised therein, is derived from cartilage. In a more particular embodiment, the cartilage is fish cartilage, and in yet more particular embodiments, the fish cartilage is shark cartilage, and the MMP inhibitor derived from fish cartilage is MDI Complex. As has been previously noted, the balance of angiogenesis-promoting and angiogenesis-suppressing factors is lost in various pathological conditions, including abnormal wound healing and skin diseases, such as eczema, acne rosacea and psoriasis (where angiogenesis plays a major role). One of the MMP inhibitors isolated from cartilage is an angiogenesis inhibitor that is also a collagenase inhibitor.

Not surprisingly, then, bovine cartilage preparations have been shown to benefit the wound healing process, and topical application of shark cartilage to wounds has accelerated the healing thereof. Also, the topical or systemic administration of cartilage, especially shark cartilage, has been suggested by a number of clinicians as a treatment for burns and various skin diseases such as psoriasis, contact dermatitis, eczema, pruritis, angiofibroma, hemangioma, and Kaposi's Sarcoma. In addition, shark and other fish cartilage is a key cosmetic ingredient in anti-aging skin formulations. Estee Lauder Inc., for example, provides such formulations. As a more specific example, MDI Complex, manufactured by Atruim Biotechnologies, Inc., Quebec, Canada, is used as the active cosmetic ingredient in various anti-aging and cosmetically restorative skin preparations. MDI Complex is a collagenase inhibitor made of fish cartilage extract.

Another example of the MMP inhibitor disclosed in embodiments of this invention is a compound having the formula shown below

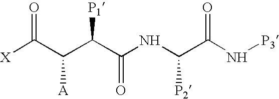

Examples of the above compound are described in U.S. Pat. No. 6,350,907, International Patent Application publication nos. WO96/33165 and WO96/33161 (to British Biotech Pharmaceuticals Ltd.), WO96/16027 (to Syntex Inc. and Agouron Pharmaceuticals Inc.), WO095/12603 (to Syntex Inc.), and by Beckett, Exp. Opin Ther. Patents, 6: 1305-1315, 1996 and Beckett et al., Drug Discovery Today, 1(1): 19-26,1996. In particular, X, A, P₁′, P₂′ and P₃′ represent chemical groups, as indicated in these publications which are herein incorporated by reference in their entirety. Various examples of the above compound are described by these publications as: 1) selective inhibitors of MMP-3 and MMP-7 relative to human fibroblast collagenase (MMP-1) and gelatinase (MMP-2); 2) inhibitors useful for matrilysin (MMP-7) inhibition; and 3) inhibitors having MMP-2/MMP-3 selectivity

Yet another example of the MMP inhibitor disclosed in embodiments of this invention is a compound having the formula shown below

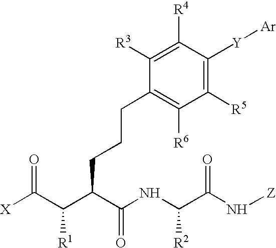

A number of examples of the above compound, their preparation, and the determination of their effectiveness and selectivity as MMP inhibitors, are disclosed and described by U.S. Pat. No. 6,350,907. In particular, X, Y, Z, Ar, R¹, R², R³, R⁴, R⁵ and R⁶ represent chemical elements or groups, as indicated in the latter reference, which is incorporated herein by reference in its entirety. Further, this reference reports, for these compounds, good activity for inhibition of MMP-3, MMP-12 and MMP-13; and good selectivity for the inhibition of MMP-3 over other MMPs such as MMPs-1, 2, 9 and 14.

As noted, the compositions of the present invention also include at least one peptide copper complex. As used herein, the term “peptide copper complex” refers to a coordination compound comprising a peptide molecule and a copper ion non-covalently complexed therewith. The peptide molecule is a chain of two or more amino acid units covalently bonded together via amide linkages (for example, —CONH—), the formation of such linkages being accompanied by the elimination of water. The amino acid units are from amino acids that are naturally occurring or otherwise. Also, at least one amide linkage nitrogen atom may have covalently bonded thereto either a hydrogen atom or another moiety.

Generally, an amino acid consists of an amino group, a carboxyl group, a hydrogen atom, and an amino acid side-chain moiety—all bonded, in the case of an alpha-amino acid, to a single carbon atom that is referred to as an alpha-carbon. The amino acid units of the peptide copper complexes comprised in compositions of the present invention may be provided by amino acids other alpha-amino acids. For example, the amino acids may be beta- or gamma-amino acids, such as those shown below.

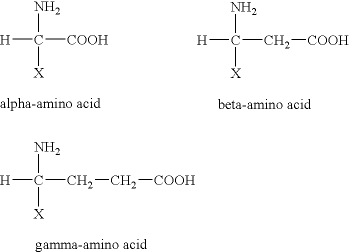

where X is the amino acid side-chain moiety.

Naturally occurring amino acids, that is, amino acids from which the amino acid units of naturally occurring proteins are derived, and their respective naturally occurring, amino acid side chain moieties, are shown below in Table 1. These naturally occurring amino acids are all in the L configuration, referring to the optical orientation of the alpha carbon or other carbon atom bearing the amino acid side chain. A peptide molecule may also comprise amino acids that are in the D optical configuration, or a mixture of amino acids where some are in the D optical configuration, and others are in the L optical configuration.

TABLE 1
NATURALLY OCCURRING AMINO ACID
SIDE-CHAIN MOIETIES

| Amino Acid Side Chain Moiety | Amino Acid |
|---|---|
| —H | Glycine |
| —CH₃ | Alanine |
| —CH(CH₃)₂ | Valine |
| —CH₂CH(CH₃)₂ | Leucine |
| —CH(CH₃)CH₂CH₃ | Isoleucine |
| —(CH₂)₄NH₃⁺ | Lysine |
| —(CH₂)₃NHC(NH₂)NH₂⁺ | Arginine |
| 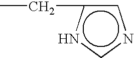 | Histidine |
| —CH₂COO— | Aspartic Acid |
| —CH₂CH₂COO— | Glutamic Acid |
| —CH₂CONH₂ | Asparagine |
| —CH₂CH₂CONH₂ | Glutamine |
| 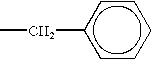 | Phenylalanine |
| 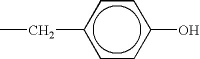 | Tyrosine |
| 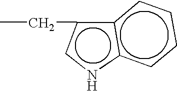 | Tryptophan |
| —CH₂SH | Cysteine |
| —CH₂CH₂SCH₃ | Methionine |
| —CH₂OH | Serine |
| —CH(OH)CH₃ | Threonine |
| 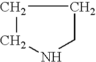 | Proline |

One example of a copper peptide complex is alanyl-histidyl-lysine:copper (II). Copper (II), as is well understood by the skilled artisan, designates a copper ion having a valence of 2 (e.g., Cu⁺²). Additional examples of the peptide copper complexes, encompassed in embodiments of the present invention, include, but are not limited to, those described in U.S. Pat. Nos. 4,665,054; 4,760,051; 4,767,753; 4,877,770; 5,023,237; 5,059,588; 5,120,831; 5,135,913; 5,145,838; 5,177,061; 5,214,032; 5,348,943; 5,538,945 and 5,550,183, incorporated herein by reference in their entireties.

Further, the expression “peptide copper complex,” as used herein, encompasses peptide copper complex derivatives. The expression “peptide copper complex derivative,” as used herein, refers to a peptide copper complex where the peptide molecule thereof has: 1) at least one amino acid side chain moiety that is a modification and/or variation of a naturally occurring, amino acid side-chain moiety; and/or 2) at least one of the hydrogens, bonded to an amide linkage nitrogen atom, substituted with a different moiety; and/or 3) the carboxyl group of the carboxyl terminal residue esterified or otherwise modified; and/or 4) at least one hydrogen, bonded to the nitrogen atom of the amino-terminal residue, substituted with a different moiety.

The amino acid side-chain moieties of the peptide copper complex derivatives may include alkyl, aryl, arylalkyl, alkoxy, or aryloxy moieties. As used herein, “alkyl” means a straight chain or branched, cyclic or noncyclic, substituted or unsubstituted, saturated or unsaturated aliphatic hydrocarbon containing from 1 to 18 carbon atoms. Representative saturated straight chain alkyls include methyl, ethyl, n-propyl and the like; while saturated branched alkyls include isopropyl, sec-butyl, isobutyl, tert-butyl, isopentyl, and the like. Representative, saturated cyclic alkyls include cyclopropyl, cyclobutyl, cyclopentyl, —CH₂cyclohexyl, and the like; while unsaturated cyclic alkyls include cyclopentenyl, cyclohexenyl, and the like. Unsaturated alkyls contain at least one double or triple bond between adjacent carbon atoms (referred to as an “alkenyl” or “alkynyl,” respectively). Representative alkenyls include ethylenyl, 1-butenyl, isobutylenyl, 2-methyl-2-butenyl, and the like; while representative alkynyls include acetylenyl, 2-butynyl, 3-methyl-1-butynyl, and the like.

Also, as used herein, “aryl” means an aromatic carbocyclic moiety such as phenyl or naphthyl, and may be substituted or unsubstituted. “Arylalkyl,” as used herein, means an alkyl having at least one alkyl hydrogen atom replaced with a substituted or unsubstituted aryl moiety, such as benzyl (i.e., —CH₂phenyl, —(CH₂)₂phenyl, —(CH₂)₃phenyl, —CH(phenyl)₂, and the like). As some examples, the amino acid side-chain moieties of alanine, valine, leucine, isoleucine and phenylalanine may generally be classified as alkyl, aryl or arylalkyl moieties.

“Alkoxy” and “aryloxy,” as used herein, refer, respectively, to alky and aryl moieties, as defined above, but each further comprising an oxygen atom used to link the moiety to the amino acid.

For example, the amino acid side-chain moieties of alanine, valine, leucine, isoleucine and phenylalanine may generally be classified as lower chain alkyl (1-12 carbon atoms), lower chain aryl (6-12 carbon atoms), or lower chain aralkyl (7-12 carbon atoms) moieties. The amino acid side-chain moieties of the peptide copper complex derivatives, may include other straight chain or branched, cyclic or noncyclic, substituted or unsubstituted, saturated or unsaturated lower chain alkyl, aryl or aralkyl moieties. Also, the peptide copper complex derivative may, for example, be N-alkylated at one or more peptide bonds; and/or its carboxyl terminus may be esterified, for example, with a methyl, ethyl, or benzyl group, or may be reduced to a hydroxy or aldehyde. Additionally, the peptide copper complex derivative may, for example, be N-alkylated, N-acylated or N-sulfonylated at the amino terminus with, for example, methyl, benzyl, acetyl, benzoyl, methanesulfonyl, or fluorenyloxycarbonyl moieties.

Examples of the peptide copper complex derivatives, encompassed in embodiments of the present invention, include, but are not limited to, those disclosed and described in the above-cited U.S. Patents that are directed to peptide copper complexes, as well as those disclosed and described in the published PCT application having the international publication number WO 94/03482, incorporated herein by reference in its entirety.

Copper is known to have many beneficial biological applications, including wound healing, treating inflammatory conditions, and effecting cosmetic improvements by, for example, stimulating a variety of processes related to skin, such as collagen, elastin and glycosaminoglycan production (see, e.g., Maquart, F. X., Pickart, L., Laurent, M., Gillery, P., Monboisse, J. C., Borel, J. P., “Stimulation of Collagen Synthesis in Fibroblast Cultures by the Tripeptide-Copper Complex Glycyl-L-Histidyl-L-Lysine-Copper(2+),” FEBS Lett. 238(2): 343-346, 1988; Wegrowski, Y., Maquart, F. X. and Borel, J. P., “Stimulation of Sulfated Glycosaminoglycan Synthesis by the Tripeptide-Copper Complex Glycyl-L-Histidyl-L-Lysine-Copper(2+),” Life Sciences 51: 1049-1056, 1992; Maguart, F. X., Bellon, G., Chaqour, B., Wegrowski, J., Patt L. M., Trachy, R. E., Monboisse, J. C., Chastang, F., Birembaut, P., Gillery, P. and Borel, J. P., “In Vivo Stimulation of Connective Tissue Accumulation by the Tripeptide-Copper Complex Glycyl-L-Histidyl-L-Lysine-Copper(2+) in Rat Experimental Wounds,” J. Clin. Invest. 92: 2368-2376, 1993). The above-cited references are incorporated herein by reference in their entireties.

Copper salts alone are ineffective, or even inhibitory, for such applications. The copper must be delivered in a biologically acceptable form. As an example, when copper is complexed with a biologically acceptable carrier molecule, such as a peptide, it may then be effectively delivered to cells.

The ability of peptide copper complexes to increase the amount of collagen in skin and to restore ECMs by, for example, stimulating the production and accumulation of collagen, elastin and glycosaminoglycan, underlies the use of peptide copper complexes, in combination with MPs and MMPs, to thereby provide the compositions of the present invention that are able to remedy abnormally destructive MP and MMP activity while repairing the damage resulting therefrom.

In certain specific embodiments of the composition of the present invention, the at least one peptide copper complex is alanyl-histidyl-lysine:copper(II) (“AHK—Cu”), valyl-histidyl-lysine:copper(II) (“VHK—Cu”), or glycyl-histidyl-lysine:copper(II) (GHK—Cu”), respectively. As is well understood in the art, copper(II) designates a copper ion having a valence of 2 (e.g., Cu⁺²). Further, such peptides may be in either the L or D form. In a related, more specific embodiment, they are all in the L form.

In another specific embodiment, the composition of the present invention includes the peptide copper complex derivative that is a derivative of GHK—Cu having the general formula:

[glycyl-histidyl-lysine-R]: copper(II)

where R is an alkyl moiety containing from 1 to 18 carbon atoms, an aryl moiety containing from 6 to 12 carbon atoms, an alkoxy moiety containing from 1 to 12 carbon atoms, or an aryloxy moiety containing from 6 to 12 carbon atoms. This derivative of GHK—Cu is further described in the above-cited U.S. Patents that are directed to peptide copper complexes.

In certain embodiments of the composition of the present invention, the concentration of the at least one peptide copper complex, by weight of the composition, ranges from about 0.01% to about 5%, from about 0.025% to about 1%, and from about 0.05% to about 0.5%, respectively. In other certain embodiments, the molar ratio of peptide to copper in the at least one peptide copper complex ranges from about 1:1 to about 3:1, and from about 1:1 to about 2:1, respectively.

The disclosed compositions of the present invention may be provided by combining at least one MP inhibitor and at least one peptide copper complex using methods that are well known to those skilled in the art. For example, an amount of dried peptide copper complex, suitable for a desired concentration, is readily dissolved in water with mixing and gentle heating. An alternative method is to prepare a solution of the desired peptide, followed by the addition of a copper salt in the desired molar ratio to yield the desired solution of the peptide copper complex. Examples of copper salts that may be used are cupric chloride and cupric acetate. When aqueous solutions of peptide copper complexes are prepared, the solutions are neutralized, typically with NaOH.

The present invention, in another representative embodiment, is also directed to a composition formed by combining at least one peptide copper complex with at least one MP inhibitor (“active compounds”), where the at least one peptide copper complex and/or the at least one MP inhibitor are encapsulated in liposomes or microsponges to aid in the delivery of the at least one peptide copper complex and/or the at least one MP inhibitor; or to increase the stability of the composition.

In yet another representative embodiment, the active compounds are formulated in an instrument adapted to deliver them to the skin via iontophoresis. As is appreciated by one skilled in the art, such a formulation is typically in the form of a liquid (i.e., solution), rather than a cream or gel. An example of an instrument adapted for such delivery is a large bandage comprising a chamber and delivering an electrical current. The chamber is situated so as to be in contact with the skin and comprises the formulation. In a related, particular embodiment, the active compounds are formulated for delivery via ultrasound. As is appreciated by one skilled in the art, ultrasound and iontophoresis enhance the delivery of the active compounds to the skin by disturbing the stratum corneum, thereby improving the transport of the active compounds.

In yet another related embodiment, a disclosed composition comprises at least one MP inhibitor and at least one peptide copper complex (“active compounds”), formulated for application to the skin after a treatment, such as laser treatment, thereof. Such treatments enhance the delivery of the components of the active compounds to the skin by removing or partially removing the stratum corneum, thereby improving the transport of the active compounds.

The compositions of the present invention, in certain embodiments, are adapted for oral or parenteral administration to a patient. Accordingly, in a particular related embodiment, the compositions further comprise an excipient, an inert and physiologically-acceptable carrier, a preservative, or a mixture thereof. Examples of suitable excipients include phosphate buffered saline, bacteriostatic saline, propylene glycol, starch, sucrose and sorbitol. Examples of suitable inert and physiologically-acceptable carriers include sterile water, physiological saline, bacteriostatic saline, and phosphate-buffered saline. Also, suitable preservatives may include, as examples, benzyl alcohol, any of the parabens, diazolidinyl urea, DMDM hydantoin, phenoxyethanol, and iodopropynyl butylcarbamate. Suitable excipients should be well tolerated, stable, and yield a consistency that allows for easy and pleasant utilization. Further, suitable preservatives should impart to the compositions of the present invention, resistance to microbial attack and toxicity to microbes.

In other certain embodiments, the compositions of the present invention are adapted for topical administration to the skin, and, accordingly, further comprise an inert and physiologically-acceptable diluent. Such compositions may also comprise a sunscreen agent, a skin conditioning agent, a skin protectant, an emollient, a humectant, a fatty alcohol, a fatty acid, an organic base, an inorganic base, a preservative, a wax ester, a steroid alcohol, a triglyceride ester, a phospholipid, a polyhydric alcohol ester, a fatty alcohol ether, a hydrophilic lanolin derivative, a hydrophilic beeswax derivative, a cocoa butter wax, a silicon oil, a pH balancer, a cellulose derivative, a hydrocarbon oil, an emulsifying agent, a surfactant, a thickening agent (a textural modifier), an excipient, or a mixture thereof.

Suitable examples of the above additional agents typically include those agents commonly used in pharmaceutical and skin care preparations. For example, suitable diluents include saline, sterile water, a petrolatum based cream, a pharmaceutically-acceptable gel, a short-chain alcohol, or a short-chain glycol. Suitable excipients and preservatives include those listed above. Thickening agents that may be used include acrylamides copolymer, carbomer, hydroxyethylcellulose, hydroxypropylcellulose, polyacrylic acid, polymethacrylic acid and polyvinyl alcohol. Suitable emulsifying agents include caprylic/capric triglyceride, ceteareth-7, cetyl alcohol, cetyl phosphate, isosteareth-11 and sodium isostearate. Examples of the above additional agents, other than those that are listed, may also be used in embodiments of this invention, as would be well appreciated by one of ordinary skill in the art.

In another embodiment, the disclosed composition is in the form of a liquid, a cream, a suspension, a gel, an emulsion, a lotion, or an oil.

In another aspect, the present invention is directed to methods for treating, in a patient, an inflammatory condition, osteoarthritis or rheumatoid arthritis, a skin disease, or aging skin, respectively. Generally, the disclosed methods comprise orally, parenterally, or topically administering to a patient in need of such treatment, a therapeutically effective amount of the composition of the present invention, the composition being suitably adapted for the mode of administration used, as described above, and as is well appreciated by one skilled in the art.

In another embodiment, directed to a method, the present invention provides a method for enhancing the wound-healing process in a patient, where the method comprises orally, parenterally, or topically administering to a patient in need of such treatment a therapeutically effective amount of the composition of the present invention, the composition again being suitably adapted for the mode of administration used, as described above, and as is well appreciated by one skilled in the art. Where the composition is administered parenterally, it is administered via at least one intravenous injection, or via at least one injection into the wound or into the area surrounding the wound.

Also disclosed are methods for, respectively, 1) stimulating hair growth on a patient; and 2) for cosmetically treating the skin of a patient to condition and smoothen the skin, reduce hyperpigmentation thereof, reduce wrinkles and fine lines therein, and otherwise reduce manifestations of environmental exposure and aging thereof. The disclosed methods comprise orally, parenterally, or locally administering to a patient in need of such treatment a therapeutically effective amount of the composition of the present invention, the composition again being suitably adapted for the mode of administration used, as described above, and as is well appreciated by one skilled in the art.

One mode of local administration of a disclosed composition for these methods is topical, where affected areas of skin are contacted with the composition, adapted for topical application as described above. As an example, a small amount of the latter (from about 1 ml to about 5 ml) may be applied to an affected area of skin from a suitable container or applicator, and, if necessary, the composition is then spread over and/or rubbed into the area of skin using the hand or finger, or a suitable device. Another mode of local administration is by intradermal injection of the composition, where the composition may comprise a vehicle suitable for such injection. One example of a suitable vehicle is sterile water.

Finally, also disclosed is a method for promoting the healing of bone in a patient, comprising administering to the affected bone, or the area surrounding the affected bone, a therapeutically effective amount of a composition of the present invention

The disclosed compositions used for the above methods may, in certain embodiments, comprise at least one peptide copper complex having a concentration, by weight of the composition, selected to be within the concentration ranges disclosed above for certain embodiments of the composition of the present invention. Also, the molar ratio of peptide to copper in the at least one peptide copper complex may, in certain other embodiments, be selected to be within the molar ratio ranges disclosed above for certain embodiments of the composition of the present invention. The concentration of the MP inhibitor will vary according to the individual type of MP inhibitor used. Typically an amount necessary to completely or partially inhibit the tissue MPs present would be used.

Also, a disclosed composition is typically packaged in a container to suit its viscosity and its use, actual or intended, by the consumer. For example, a lotion or fluid cream may be packaged in a bottle, roll-ball applicator, capsule, propellant-driven aerosol device, or a container fitted with a manually operated pump. A cream can simply be stored in a non-deformable bottle or squeeze container, such as a tube or a lidded jar.

The following examples are provided for the purpose of illustration, not limitation.

### EXAMPLE 1

### A Representative Moisturizing Lotion

| | Preferred | |
|---|---|---|
| Ingredients | weight % | Range |
| Water | 73.82% | 50% to 80% |
| Glycerin | 1.00% | 0.01% to 25% |
| xanthan gum | 0.50% | 0.01% to 25% |
| diisopropyl adipate | 4.00% | 0.01% to 25% |
| isocetyl stearate | 6.00% | 0.01% to 25% |
| octyl palmitate | 10.00% | 0.01% to 25% |
| glyceryl stearate | 1.00% | 0.01% to 10% |
| cetyl alcohol | 1.00% | 0.01% to 10% |
| stearyl alcohol | 0.80% | 0.01% to 10% |
| behenyl alcohol | 0.50% | 0.01% to 10% |
| palmitic acid | 0.30% | 0.01% to 10% |
| stearic acid | 0.25% | 0.01% to 10% |
| glycyl-L-histidyl-L-lysine copper complex | 0.20% | 0.01% to 10% |
| TIMP-1 | 0.03% | 0.001% to 10% |
| Propylene glycol | 0.55% | 0.001% to 10% |
| diazolidinyl urea | 0.03% | 0.001% to 10% |
| iodopropynyl butylcarbonate | 0.02% | 0.001% to 10% |
| Total | 100.00% | |

This formulation is beneficial in that the MP inhibitor provides protease inhibitory action to the skin, in addition to the anti-inflammatory and tissue rebuilding activity provided by the peptide copper compound. Such a formulation would sooth, protect, and restore the youthful appearance of the skin, lost due to enhanced MP activity and decreased synthetic activity of the ECM.

### EXAMPLE 2

### A Representative Moisturizing Cream

| | Preferred | |
|---|---|---|
| Ingredients | weight % | Range |
| Purified water | 76.35% | 50% to 80% |
| ethylhexyl palmitate | 8.00% | 0.01% to 25% |
| Octyldodecanol | 2.50% | 0.01% to 25% |
| butyloctyl calicylate | 2.00% | 0.01% to 25% |
| Squalane | 1.50% | 0.01% to 25% |
| jojoba oil | 0.50% | 0.01% to 10% |
| tocopheryl acetate | 0.20% | 0.01% to 10% |
| Bisabolol | 0.20% | 0.01% to 10% |
| Polyacrylamide | 1.50% | 0.01% to 10% |
| Laureth-7 | 0.50% | 0.01% to 10% |
| Glycerin | 3.00% | 0.01% to 25% |
| Panthenol | 0.60% | 0.01% to 10% |
| Allantoin | 0.10% | 0.01% to 10% |
| Cyclomethicone | 0.50% | 0.01% to 10% |
| Carbomer | 0.10% | 0.01% to 10% |
| polysorbate 20 | 0.20% | 0.01% to 10% |
| glycyl-L-histidyl-L-lysine copper | 0.25% | 0.01% to 5% |
| complex | | |
| α₂-Macroglobulin | 1.00% | 0.001% to 10% |
| propylene glycol | 0.56% | 0.001% to 10% |
| diazolidinyl urea | 0.30% | 0.001% to 10% |
| Methylparaben | 0.11% | 0.001% to 10% |
| Propylparaben | 0.03% | 0.001% to 10% |
| Total | 100.00% | |

This formulation is beneficial in that the MP inhibitor provides protease inhibitory action to the skin, in addition to the anti-inflammatory and tissue rebuilding activity provided by the peptide copper compound. Such a formulation would sooth, protect, and restore the youthful appearance of the skin, lost due to enhanced MP activity and decreased synthetic activity of the ECM.

### EXAMPLE 3

### A Representative Body Lotion

| | Preferred | |
|---|---|---|
| Ingredients | weight % | Range |
| Water | 74.35% | 50% to 80% |
| hydrogenated vegetable oil | 6.00% | 0.01% to 25% |
| canola oil | 4.00% | 0.01% to 25% |
| polyoxyethylene stearyl stearate | 4.00% | 0.01% to 25% |
| Steareth-21 | 2.00% | 0.01% to 25% |
| Octyldodecanol | 6.00% | 0.01% to 25% |
| sorbeth-30 | 2.50% | 0.01% to 25% |
| glycyl-L-histidyl-L-lysine copper complex | 0.10% | 0.01% to 10% |
| Shark cartilage extract | 0.20% | 0.001% to 10% |
| Phenoxyethanol | 0.56% | 0.001% to 10% |
| Chlorphenesin | 0.16% | 0.001% to 10% |
| Methylparaben | 0.07% | 0.001% to 10% |
| Benzoic Acid | 0.06% | 0.001% to 10% |
| Total | 100.00% | |

This formulation is beneficial in that the MP inhibitor provides protease inhibitory action to the skin, in addition to the anti-inflammatory and tissue rebuilding activity provided by the peptide copper compound. Such a formulation would sooth, protect, and restore the youthful appearance of the skin, lost due to enhanced MP activity and decreased synthetic activity of the ECM.

### EXAMPLE 4

### A Representative Hair Treatment Composition

| | Preferred | |
|---|---|---|
| Ingredients | weight % | Range |
| Water | 96.99% | 50% to 80% |
| Sodium Chloride | 0.9% | 0.01% to 25% |
| L-alanyl-L-histidyl-L-lysine copper | 0.20% | 0.01% to 10% |
| complex | | |
| MDI Complex | 1.00% | |
| propylene glycol | 0.56% | 0.001% to 10% |
| Phenoxyethanol | 0.30% | 0.001% to 10% |
| Isopropylparaben | 0.02% | 0.001% to 10% |
| Isobutylparaben | 0.03% | 0.001% to 10% |
| Total | 100.00% | |

This formulation is beneficial in that the MP inhibitor provides protease inhibitory action to the hair follicle and surrounding skin, in addition to the hair growth activity provided by the peptide copper compound. Such a formulation would enhance the health of the scalp and promote the growth of hair.

### EXAMPLE 5

The efficacy of the disclosed compositions of this invention can be demonstrated via standard assays used to assess the performance of such compositions. For example, the compositions of this invention can be provided to volunteer subjects having signs of photo damaged skin such as age spots, hyperpigmentation, fine lines and wrinkles. These signs of clinical aging could be rated using, for example, a scale of 0-9 at baseline, and at weeks 4 and 8. Subjects could be given compositions suitable for topical application, formulated according to the present invention, along with instructions that the compositions are to be topically applied twice daily to the areas showing signs of photodamage and aging. Clinical photographs may also be taken for comparison.

At the end of 4 and 8 weeks, the clinical signs of aging would again be assessed, and corresponding photographs taken for comparison with those taken earlier and subsequently. Comparison of data with the data collected earlier and subsequently would reveal a diminishment of the clinical signs of aging and photodamaged skin as a result of the treatment with the composition with the skin care compositions and preparations of this invention.

All of the U.S. patents, U.S. patent application publications, U.S. patent applications, foreign patents, foreign patent applications and non-patent publications listed in the Application Data Sheet, are incorporated herein by reference in their entirety.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A composition comprising at least one peptide copper complex and at least one metalloproteinase inhibitor.

2. The composition of claim 1 wherein the at least one metalloproteinase inhibitor is a matrix metalloproteinase inhibitor.

3. The composition of claim 2 wherein the matrix metalloproteinase inhibitor is a naturally produced tissue inhibitor of metalloproteinase, a recombinant tissue inhibitor of metalloproteinase, or a mutant thereof.

4. The composition of claim 3 wherein the tissue inhibitor of metalloproteinase is TIMP-1.

5. The composition of claim 3 wherein the tissue inhibitor of metalloproteinase is TIMP-2.

6. The composition of claim 3 wherein the tissue inhibitor of metalloproteinase is TIMP-3.

7. The composition of claim 3 wherein the tissue inhibitor of metalloproteinase is TIMP4.

8. The composition of claim 2 wherein the matrix metalloproteinase inhibitor is naturally occurring α₂-macroglobulin.

9. The composition of claim 2 wherein the matrix metalloproteinase inhibitor is a non-peptidic hydroxamate inhibitor or a pipcolinic hydroxamic acid derivative.

10. The composition of claim 9 wherein the non-peptidic hydroxamate inhibitor is marimastat.

11. The composition of claim 9 wherein the pipcolinic hydroxamic acid derivative is pipcolinic sulfamide.

12. The composition of claim 2 wherein the matrix metalloproteinase inhibitor is a malony-α-mercaptoalcohol, a succinyl-α-mercaptoalcohol, a malony-α-mercaptoketone, or a succinyl-α-mercaptoketone.

13. The composition of claim 2 wherein the matrix metalloproteinase inhibitor is a naturally occurring macrocyclic lactone.

14. The composition of claim 2 wherein the matrix metalloproteinase inhibitor is a bisphosphonate.

15. The composition of claim 2 wherein the matrix metalloproteinase inhibitor is an antibiotic.

16. The composition of claim 15 wherein the antibiotic is anthracycline, tetracycline, doxycycline, minocycline, or a derivative thereof.

17. The composition of claim 2 wherein the matrix metalloproteinase inhibitor is a retinoid, a thyroid hormone, a glucocorticoid, progesterone or an androgen.

18. The composition of claim 2 wherein the matrix metalloproteinase inhibitor is a peroxisome proliferator-activated receptor gamma.

19 The composition of claim 2 wherein the matrix metalloproteinase inhibitor is an antisense RNA or ribozyme.

20. The composition of claim 2 wherein the matrix metalloproteinase inhibitor is obtained from cartilage.

21. The composition of claim 20 wherein the cartilage is fish cartilage.

22. The composition of claim 21 wherein the matrix metalloproteinase inhibitor is MDI Complex.

23. The composition of claim 21 wherein the fish cartilage is shark cartilage.

24. The composition of claim 1 wherein the at least one peptide copper complex is L-alanyl-L-histidyl-L-lysine:copper(II), L-valyl-L-histidyl-L-lysine:copper(II) or glycyl-L-histidyl-L-lysine:copper(II).

25. The composition of claim 1 wherein the at least one peptide copper complex is alanyl-histidyl-lysine:copper(II).

26. The composition of claim 1 wherein the at least one peptide copper complex is valyl-histidyl-lysine:copper(II).

27. The composition of claim 1 wherein the at least one peptide copper complex is glycyl-histidyl-lysine:copper(II).

28. The composition of claim 1 wherein the at least one peptide copper complex is [glycyl-histidyl-lysine-R]:copper(II), wherein R is an alkyl moiety containing from one to eighteen carbon atoms, an aryl moiety containing from six to twelve carbon atoms, an alkoxy moiety containing from one to twelve carbon atoms, or an aryloxy moiety containing from six to twelve carbon atoms

29. The composition of claim 1 wherein the concentration of the at least one peptide copper complex, as a percentage of the total weight of the composition, ranges from about 0.01% to about 5%.

30. The composition of claim 1 wherein the concentration of the at least one peptide copper complex, as a percentage of the weight of the composition, ranges from about 0.025% to about 1.0%.

31. The composition of claim 1 wherein the concentration of the at least one peptide copper complex, as a percentage of the weight of the composition, ranges from about 0.05% to about 0.5%.

32. The composition of claim 1 wherein the molar ratio of peptide to copper in the at least one peptide copper complex ranges from about 1:1 to about 3:1.

33. The composition of claim 1 wherein the molar ratio of peptide to copper in the at least one peptide copper complex ranges from about 1:1 to about 2:1.

34. The composition of claim 1 wherein the at least one metalloproteinase inhibitor and/or the at least one peptide copper complex are encapsulated in a liposome or microsponge adapted to aid in the delivery of the at least one metalloproteinase inhibitor and/or at least one peptide copper complex to the skin of a patient, or to enhance the stability of the composition.

35. The composition of claim 1 wherein the at least one metalloproteinase inhibitor and the at least one peptide copper complex are formulated in an instrument adapted to deliver the same via iontophoresis to the skin of a patient.

36. The composition of claim 1 wherein the at least one metalloproteinase inhibitor and the at least one peptide copper complex are formulated for delivery to the skin of a patient, where the delivery is enhanced by ultrasound.

37. The composition of claim 1 wherein the at least one metalloproteinase inhibitor and the at least one peptide copper complex are formulated for application to the skin after a treatment to remove or partially remove the stratum corneum thereof.

38. The composition of claim 1, further comprising an excipient, an inert and physiologically-acceptable carrier, a preservative, or a mixture thereof.

39. The composition of claim 1, further comprising an inert and physiologically-acceptable diluent.

40. The composition of claim 39, further comprising a sunscreen agent, a skin-conditioning agent, a skin protectant, an emollient, a humectant, an emulsifying agent, a thickening agent, or a mixture thereof.

41. The composition of claim 40, further comprising a fatty alcohol, a fatty acid, an organic base, an inorganic base, a wax ester, a steroid alcohol, a triglyceride ester, a phospholipid, a polyhydric alcohol ester, a fatty alcohol ether, a hydrophilic lanolin derivative, a hydrophilic beeswax derivative, a cocoa butter wax, a silicon oil, a pH balancer, a cellulose derivative, a hydrocarbon oil, a surfactant, or a mixture thereof.

42. The composition of claim 1 wherein the composition is in the form of a liquid, a cream, a suspension, a gel, an emulsion, a lotion, or an oil.

43. A method for treating an inflammatory condition in a patient, comprising orally, parenterally, or topically administering to a patient in need of such treatment, a therapeutically effective amount of the composition of claim 1.

44. A method for treating osteoarthritis or rheumatoid arthritis in a patient comprising orally, parenterally, or topically administering to a patient in need of such treatment, a therapeutically effective amount of the composition of claim 1.

45. A method for enhancing the wound-healing process in a patient, comprising orally, parenterally, or topically administering to a patient in need thereof, a therapeutically effective amount of the composition of claim 1.

46. The method of claim 45 wherein the parenteral administration of the composition of claim 1 is at least one intravenous injection or at least one injection into the wound or into the area surrounding the wound.

47. A method for treating a skin disease comprising orally, parenterally, or topically administering to a patient in need of such treatment, a therapeutically effective amount of the composition of claim 1.

48. A method for cosmetically treating skin, comprising orally or parenterally administering to a patient in need of such treatment a therapeutically effective amount of the composition of claim 1.

49. The method of claim 48 wherein the cosmetic treatment of the skin is smoothening the skin, reducing hyperpigmentation of the skin, reducing wrinkles and fine lines in the skin, reducing evidence of photodamage of the skin, or reducing the signs of aging in the skin.

50. A method for stimulating the growth of hair in a patient, comprising orally, parenterally, or locally administering to a patient in need thereof, a therapeutically effective amount of the composition of claim 1.

51. The method of claim 50 wherein the local administration is by topical application or by intradermal injection.

52. A method for promoting the healing of affected bone in a patient, comprising administering to the affected bone, or the area surrounding the affected bone, a therapeutically effective amount of the composition of claim 1.
